(19) United States
(12) Patent Application Publication
(10) Pub. No.: US 2025/0051536 A1
(43) Pub. Date: Feb. 13, 2025

(54) METHOD OF RECYCLING POLYESTER FABRIC

(71) Applicant: NAN YA PLASTICS CORPORATION, TAIPEI (TW)

(72) Inventors: Te-Chao Liao, TAIPEI (TW); Jung-Jen Chuang, TAIPEI (TW); Yu Ti Tseng, TAIPEI (TW); Zhang-Jian Huang, TAIPEI (TW)

(73) Assignee: NAN YA PLASTICS CORPORATION, TAIPEI (TW)

(21) Appl. No.: 18/467,701

(22) Filed: Sep. 14, 2023

(30) Foreign Application Priority Data
Aug. 8, 2023 (TW) 112129737

(51) Int. Cl. C08J 11/24; C08G 63/183; C08J 11/02

(52) U.S. Cl. CPC C08J 11/24; C08G 63/183; C08J 11/02; C08J 2367/02

(57) ABSTRACT

A method of recycling polyester fabric includes following steps. A polyester fabric including polyethylene terephthalate and dyes is provided. An extraction process including using ethylene glycol as an extraction solvent, immersing the polyester fabric in the extraction solvent, and extracting under a temperature of 80° C. to 180° C. is performed to remove the dyes from the polyester fabric. A depolymerization process including using a chemical depolymerization solution to depolymerize the polyester fabric treated with the extraction process to obtain a product including BHET is performed. The chemical depolymerization solution is ethylene glycol. A purification process is performed to remove impurities of the product obtained by the depolymerization process and to obtain a purified BHET. A solvent recycling process is performed. The solvent recycling process includes collecting waste solutions of the extraction process and the depolymerization process, and recycling ethylene glycol in the waste solutions by a distillation separating system.

### CROSS-REFERENCE TO RELATED APPLICATION

This application claims the priority benefit of Taiwan application serial no. 112129737, filed on Aug. 8, 2023. The entirety of the above-mentioned patent application is hereby incorporated by reference herein and made a part of this specification.

### BACKGROUND OF THE INVENTION

### Field of the Invention

The invention relates to a method of recycling a fabric, and in particular to a method of recycling a polyester fabric.

### Description of Related Art

The recycling method of waste polyester fabrics (PET fabric) is usually to depolymerize the polyester fabric using chemical depolymerization liquid to obtain products including bis(2-hydroxyethyl) terephthalate (BHET). Bishydroxyethyl terephthalate may be repolymerized to obtain recycled polyester fabric (r-PET). Waste polyester fabrics are usually further subjected to processes such as dying and used as clothing, for example. In order to obtain high quality recycled polyester fabrics (r-PET) from these dyed waste polyester fabrics, impurities (such as dyes, etc.) in the polyester fabrics need to be removed before and/or after the depolymerization reaction in order to obtain purer bishydroxyethyl terephthalate, so as to be polymerized and recycled. Therefore, how to recycle the polyester fabrics in an efficient manner is an issue that needs improvement at present.

### SUMMARY OF THE INVENTION

The invention provides a method of recycling a polyester fabric having a simplified process that may reduce production costs.

The method of recycling the polyester fabric of the invention includes the following steps. A polyester fabric is provided. The polyester fabric includes polyethylene terephthalate and dyes. An extraction process is performed to remove the dyes from the polyester fabric. The extraction process includes using ethylene glycol as an extraction solvent, immersing the polyester fabric in the extraction solvent, and extracting under a temperature of 80° C. to 180° C. A depolymerization process is performed. The depolymerization process includes using a chemical depolymerization solution to depolymerize the polyester fabric treated with the extraction process to obtain a product including BHET (bis(2-hydroxyethyl terephthalate)). The chemical depolymerization solution is ethylene glycol. A purification process is performed to remove impurities of the product obtained by the depolymerization process and to obtain a purified BHET. A solvent recycling process is performed. The solvent recycling process includes collecting waste solutions of the extraction process and the depolymerization process, and recycling ethylene glycol in the waste solutions by a distillation separating system.

In an embodiment of the invention, in the extraction process, the polyester fabric is subjected to 1 to 10 extraction steps, and each of the extraction steps is to soak the polyester fabric in a new extraction solvent.

In an embodiment of the invention, a time of each of the extraction steps is between 20 minutes and 1 hour.

In an embodiment of the invention, in each of the extraction steps, a weight ratio of the extraction solvent to the polyester fabric is 5 to 10.

In an embodiment of the invention, the depolymerization process is performed under a temperature of 190° C. to 240° C.

In an embodiment of the invention, a weight of the chemical depolymerization liquid accounts for 30 wt % to 80 wt % of a total weight of the chemical depolymerization liquid and the polyester fabric treated with the extraction process.

In an embodiment of the invention, the depolymerization process is performed in an environment containing a catalyst, and the catalyst includes an organic metal or an ionic liquid.

In an embodiment of the invention, a weight of the catalyst accounts for 0.3 wt % to 8 wt % of a total weight of the chemical depolymerization liquid and the polyester fabric treated with the extraction process.

In an embodiment of the invention, a time of the depolymerization process is between 1 hour and 6 hours.

In an embodiment of the invention, in the solvent recycling process, the recycling rate of ethylene glycol is greater than 98 wt %.

Based on the above, the method of recycling the polyester fabric of the invention may simplify the process and obtain r-PET having good hue quality and yield by using ethylene glycol as the extraction solvent and the chemical depolymerization solution. Moreover, the method of recycling the polyester fabric of the invention may effectively utilize a single distillation separation system to recycle the extraction solvent and the chemical depolymerization liquid, thus not only reducing the loss of solvent, but also reducing the cost of equipment and operations.

### DESCRIPTION OF THE EMBODIMENTS

Hereinafter, embodiments of the invention are described in detail. However, these embodiments are illustrative, and the disclosure of the invention is not limited thereto.

Herein, a range indicated by “one value to another value” is a general representation which avoids enumerating all values in the range in the specification. Therefore, the record of a specific numerical range covers any number within this numerical range and any smaller numerical range bounded by any number within that numerical range as if such any number and such smaller numerical ranges were expressly written in the specification.

The following are specific examples to illustrate the implementation of the invention. Those skilled in the art may understand the advantages and effects of the invention from the content disclosed in this specification. The invention may be implemented or applied through other different specific embodiments, and various modifications and changes may be made to the details in this specification based on different viewpoints and applications without departing from the concept of the invention. The following embodiments further describe the relevant technical content of the invention in detail, but the disclosed content is not intended to limit the scope of the invention. In addition, the term “or” used herein may include any one or a combination of a plurality of the associated listed items depending on the actual situation.

The method of recycling the polyester fabric of an embodiment of the invention is described in detail below.

First, a polyester fabric is provided. The polyester fabric includes polyethylene terephthalate (PET) and dyes. In some embodiments, polyethylene terephthalate accounts for more than 80 wt % of the polyester fabric, but the invention is not limited thereto. In some embodiments, the dyes occupy more than 0.1 wt % of the polyester fabric, but the invention is not limited thereto.

Then, an extraction process is performed to remove the dyes from the polyester fabric. The extraction process includes, for example, using ethylene glycol as an extraction solvent, immersing the polyester fabric in the extraction solvent, and performing extraction under a temperature of 80° C. to 180° C.

In some embodiments, during the extraction process, the polyester fabric may be subjected to a plurality of extraction steps, such as 1 to 10, 3 to 6, or other suitable number, with the polyester fabric being immersed in a new extraction solvent for each of the extraction steps.

In some embodiments, the time of each of the extraction steps is between 20 minutes and 1 hour, but the invention is not limited thereto.

In some embodiments, in each of the extraction steps, the weight ratio of the extraction solvent to the polyester fabric is 5 to 10. In this way, efficient extraction may be achieved while taking energy consumption and operation cost into consideration, but the invention is not limited thereto.

Next, a depolymerization process is performed. The depolymerization process includes using a chemical depolymerization solution to depolymerize the polyester fabric treated with the extraction process to obtain a product including BHET (bis(2-hydroxyethyl terephthalate)). The chemical depolymerization solution is ethylene glycol. Since the extraction solvent is the same as the chemical depolymerization solution and both are ethylene glycol, the polyester fabric after the extraction process in the present embodiment may be directly subjected to the depolymerization process without going through a drying procedure to remove the extraction solvent before performing the depolymerization process. The process steps are simplified and the loss of the extraction solvent through the drying process may be avoided.

In some embodiments, the product obtained by the depolymerization process further includes an oligomer.

In some embodiments, the chemical depolymerization solution accounts for 30 wt % to 80 wt % of the total weight of the chemical depolymerization solution and the polyester fabric treated with the extraction process, preferably 40 wt % to 70 wt %, but the invention is not limited thereto.

In some embodiments, the depolymerization process is performed in an environment containing a catalyst to increase the reaction rate. The catalyst may include organic metal or ionic liquid. Examples of the organic metal include zinc acetate, organic titanium, organic antimony, organic aluminum, or other suitable organic metals. The ionic liquid is selected from, for example, at least one of the material group consisting of 1-butyl-3-methylimidazolium hexafluorophosphate (BMI-PF6), 1-butyl-3-methylimidazolium tetrachlorozincate (BMI2ZnCl4), 1-butyl-3-methylimidazolium tetrachloroironate (BMI2FeCl4), 1-butyl-3-methylimidazolium tetrachlorocobaltate (BMI2CoCl4), and 1-butyl-3-methylimidazolium tetrafluoroborate (BMI-BF4), but the invention is not limited thereto. In some embodiments, the ionic liquid may be grafted onto the particulate substrate to facilitate recycling of the ionic liquid. The particulate substrate may include carbon, silicon, iron, nickel, cobalt, or other suitable substrates.

In some embodiments, the catalyst accounts for 0.3 wt % to 8 wt % of the total weight of the chemical depolymerization solution and the polyester fabric treated with the extraction process, preferably 1.0 wt % to 5.0 wt %, but the invention is not limited thereto.

In some embodiments, the time of the depolymerization process is between 1 hour and 6 hours, preferably between 2 hours and 4 hours, but the invention is not limited thereto.

Next, a purification process is performed to remove impurities of the product obtained by the depolymerization process and to obtain a purified BHET. For example, the purification process may include dissolving the product obtained by the depolymerization process in water, cooling the product to crystallize the ethylene terephthalate, and filtering.

In some embodiments, the purification process may also include using diatomaceous earth, activated carbon, acidic clay, etc. to adsorb impurities remaining in the product obtained from the depolymerization process.

In some embodiments, the purification process may further include drying the filtered ethylene terephthalate through a procedure such as hot air, microwave, or infrared rays.

Next, the purified polyethylene terephthalate may be subjected to a polymerization process to form recycled polyethylene terephthalate. The recycled polyethylene terephthalate produced by the recycling method of the present embodiment has good hue quality, for example, the L value is between 55 and 65, the a value is between −1 and +1, and the b value is between −5 and 5, but the invention is not limited thereto. The L value, the a value, and the b value refer to the CIE Lab color space commonly used in the industry. The L value represents the brightness, the a value represents the green-red value, and the b value represents the blue-yellow value.

In some embodiments, recycled polyester pellets may be formed by pelletizing the polymerized recycled polyethylene terephthalate through a single-screw pelletizer or a twin-screw pelletizer.

In addition, a solvent recycling process may be performed after the extraction process and the depolymerization process. The solvent recycling process includes concentrating the waste solutions produced by the extraction process and the depolymerization process, and recycling the ethylene glycol in the waste solutions through a distillation separation system. Since the extraction solvent and chemical depolymerization solution both use ethylene glycol, the ethylene glycol may be separated and recycled through a single distillation separation system, without the need to separately distill the extraction solvent and the chemical depolymerization liquid. In this way, equipment costs may be reduced and the operating procedures for solvent recycling may be reduced.

In some embodiments, in the solvent recycling process, the recycling rate of ethylene glycol is greater than 98 wt % to effectively recycle ethylene glycol and reduce the loss rate of ethylene glycol (that is, the loss rate of ethylene glycol is less than 2 wt %).

In some embodiments, the distillation separation system includes a dehydration tower and a glycol purification tower.

Hereinafter, the method of recycling the polyester fabric of the invention is described in detail by means of experimental examples. However, the following experimental examples are not intended to limit the invention.

### Example 1

106 grams of PET fabric (which contains impurities such as about 100.2 grams of PET, about 5.8 grams of dyes) was taken and placed into a 1 L beaker, 500 g of ethylene glycol (EG) (extraction solvent) was added, and then the mixture was heated to 135° C. and maintained for 30 minutes. Then the liquid was filtered out with a funnel having a pore size of 1 mm, the wet PET fabric contained about 50 grams of EG, and then the wet PET fabric was put into a 1 L beaker, 450 grams of EG (extraction solvent) was added and the mixture was heated to 135° C. and maintained for 30 minutes, and the process was repeated twice. That is, the PET textile underwent a total of 4 extraction steps.

A total of about 149.9 grams of the wet PET fabric after 4 extractions contained impurities such as about 99.8 grams of PET fabric, about 50 grams of EG, and about 0.1 gram of dyes. The wet PET fabric was placed into a 1 L reaction device (containing heating pack, formed by stirring and condensation), 350 grams of EG (chemical depolymerization solution) and 1 gram of zinc acetate (catalyst) were added, the temperature was raised to 198° C. and maintained for 4 hours, and a depolymerization process was performed to depolymerize PET into crude BHET product.

The crude BHET product was cooled to 15° C. to crystallize and precipitate BHET, and then filtered to obtain 200 grams of BHET filter cake. 200 grams of BHET filter cake was placed into a 1 L beaker, 200 grams of water was added, and the temperature was raised to 90° C. to dissolve BHET, then 5 grams of activated carbon was added and stirring was performed for 1 hr, then impurities such as activated carbon was filtered out with 0.1 μm filter paper, then the filtrate was cooled from 90° C. to 5° C. to precipitate BHET crystals, and then filtering was performed with 1 μm filter paper to obtain a BHET filter cake, and then drying was performed under the conditions of 75° C. and 100 torr to obtain about 120 g of purified BHET.

120 grams of purified BHET was polymerized to obtain about 88.7 grams of recycled PET (r-PET) and 31.3 grams of by-products such as EG and oligomers. r-PET yield=88.7 g/100.2 g=88.5%, hue quality L=60.4, a=0.2, b=1.2.

The waste solutions obtained from the extraction process and the depolymerization process were recycled through the distillation process. The recycling rate of EG=2,167 grams/2,200 grams=98.5%, with a total loss of about 33 grams of EG.

### Example 2 to Example 6

The processing method of Example 2 to Example 6 is similar to Example 1. But the processing conditions (including extraction temperature, extraction time, depolymerization temperature, depolymerization time) of Example 2 to Example 6 are as shown in Table 1. The L, a, b, and yields of the r-PET obtained in Example 2 to Example 6 and the total loss rate of the extraction solvent and the chemical depolymerization solution are recited in Table 1.

TABLE 1

| | | | Example 1 | Example 2 | Example 3 | Example 4 | Example 5 | Example 6 |
|---|---|---|---|---|---|---|---|---|
| Process | Extraction solvent | | EG | | | | | |
| conditions | Extraction temperature (° C.) | | 135 | 135 | 125 | 125 | 150 | 150 |
| | Extraction time (minutes) | | 30 | 30 | 40 | 40 | 20 | 20 |
| | Depolymerization | | 198 | 190 | 205 | 210 | 220 | 240 |
| | temperature (° C.) | | | | | | | |
| | Depolymerization time | | 4.0 | 6.0 | 3.5 | 3.0 | 2.0 | 1.0 |
| | (hours) | | | | | | | |
| Quality | r-PET | L | 58.4 | 56.7 | 60.2 | 61.0 | 61.9 | 62.7 |
| | | a | 0.2 | 0.4 | 0.1 | −0.2 | 0.2 | 0.7 |
| | | b | 2.7 | 2.5 | 2.9 | 3.1 | 3.8 | 4.6 |
| | | Yield (%) | 88.5 | 87.3 | 85.4 | 86.2 | 84.3 | 81.2 |

| | | | | | | | |
|---|---|---|---|---|---|---|---|
| Solvent | Total loss of extraction | 33.0 | 31.8 | 35.4 | 33.5 | 32.7 | 36.9 |
| recycling | solvent and chemical | | | | | | |
| | depolymerization solution | | | | | | |
| | (g) | | | | | | |
| | Total loss rate of extraction | 1.5 | 1.4 | 1.6 | 1.5 | 1.5 | 1.7 |
| | solvent and chemical | | | | | | |
| | depolymerization solution | | | | | | |
| | (%) | | | | | | |

### Comparative Example 1

106 grams of PET fabric (which contained impurities such as about 100.2 grams of PET, about 5.8 grams of dyes) was taken and placed into a 1 L beaker, 500 g of xylene (extraction solvent) was added, and then the mixture was heated to 135° C. and maintained for 30 minutes. Then the liquid was filtered out with a funnel having a pore size of 1 mm, the wet PET fabric contained 50 grams of xylene, and then the wet PET fabric was put into a 1 L beaker, 450 grams of xylene (extraction solvent) was added and the mixture was heated to 135° C. and maintained for 30 minutes, and the process was repeated twice. That is, the PET textile underwent a total of 4 extraction steps.

After 4 extractions, a total of about 149.5 grams of wet PET fabric (which contained impurities such as about 99.4 grams of PET fabric, about 50 grams of xylene, and about 0.1 grams of dyes) needed to be dried (at 100 torr, 120° C.) to remove xylene, so as to obtain about 99.5 grams of dry PET fabric (which included impurities such as about 99.2 grams of PET, 0.2 grams of xylene, and 0.1 grams of dyes), and about 50 grams of xylene was collected by condensation at 5° C.

The 99.5 grams of dry PET fabric was placed into a 1 L reaction device (containing heating pack, formed by stirring and condensation), 400 grams of EG (chemical depolymerization solution) and 1 gram of zinc acetate (catalyst) were added, the temperature was raised to 198° C. and maintained for 4 hours, and a depolymerization reaction was performed to depolymerize PET into crude BHET product.

The crude BHET product was cooled to 15° C. to precipitate BHET crystals, and then 200 g of BHET filter cake was filtered out. 200 grams of BHET filter cake was placed into a 1 L beaker, 200 grams of water was added, and the temperature was raised to 90° C. to dissolve BHET, then 5 grams of activated carbon was added and stirring was performed for 1 hr, then impurities such as activated carbon was filtered out with 0.1 μm filter paper, and the filtrate was cooled from 90° C. to 5° C. to precipitate BHET crystals, and then filtering was performed with 1 μm filter paper to obtain a BHET filter cake, and then drying was performed at 75° C. and 100 torr to obtain about 115.0 g of purified BHET.

115.0 grams of purified BHET was polymerized to obtain 86.3 grams of recycled PET (r-PET) and 28.7 grams of by-products such as EG and oligomers. r-PET yield=86.3 g/100.2 g=86.1%, hue quality L=60.7, a=0.1, b=3.2.

Recycling of extraction solvent: approximately 1,631 grams of the extraction solvent was recycled by distillation and added in the 50 grams of extraction solvent collected in the drying process, and a total of 1,681 grams of xylene was collected. Xylene yield= 1,681/1,850=90.9%, 169 grams of xylene solvent was leaked, and the loss rate was 9.1%.

Recycling of the chemical depolymerization liquid: 394 grams of EG in the waste solutions of the depolymerization process was recycled by distillation. The recycling rate of EG=394 grams/400 grams=98.5%. 6 grams of EG solvent was leaked, and the loss rate was 1.5%.

The total loss of the extraction solvent and chemical depolymerization solution was 175 grams, and the total loss rate thereof was 7.8%.

### Comparative Example 2 to Comparative Example 6

The processing method of Comparative example 2 to Comparative example 6 is similar to Comparative example 1. But the processing conditions (including extraction solvent, extraction temperature, extraction time, depolymerization temperature, depolymerization time) of Comparative example 2 to Comparative example 6 are as shown in Table 2. The L, a, b, and yields of the r-PET obtained in Comparative example 2 to Comparative example 6 and the loss rate of the extraction solvent and the chemical depolymerization solution are recited in Table 2.

TABLE 2

| | | | Comparative | Comparative | Comparative | Comparative | Comparative | Comparative |
|---|---|---|---|---|---|---|---|---|
| | | | example 1 | example 2 | example 3 | example 4 | example 5 | example 6 |
| Process | Extraction solvent | | Xylene | Xylene | Propylene | Propylene | Dimethyl | Dimethyl |
| conditions | | | | | glycol | glycol | Acetamide | Acetamide |
| | | | | | Monomethyl | Monomethyl | | |
| | | | | | ether | ether | | |
| | Extraction temperature (° C.) | | 135 | 135 | 135 | 135 | 135 | 135 |
| | Extraction time (minutes) | | 30 | 30 | 40 | 40 | 20 | 20 |
| | Depolymerization | | 198 | 190 | 205 | 210 | 220 | 240 |
| | temperature (° C.) | | | | | | | |
| | Depolymerization time | | 4.0 | 6.0 | 3.5 | 3.0 | 2.0 | 1.0 |
| | (hours) | | | | | | | |
| Quality | r-PET | L | 60.7 | 59.4 | 58.7 | 59.3 | 60.2 | 60.1 |
| | | a | 0.1 | 0.2 | −0.1 | −0.2 | 0.3 | 0.1 |
| | | b | 3.2 | 2.9 | 3.4 | 3.9 | 4.2 | 4.8 |
| | | Yield | 86.1 | 85.8 | 85.4 | 85.3 | 83.4 | 80.2 |
| | | (%) | | | | | | |
| Solvent | Loss of | Quantity | 169 | 168 | 162 | 166 | 172 | 174 |
| recycling | extraction | (g) | | | | | | |
| | solvent | Ratio | 9.1 | 9.1 | 8.8 | 9.0 | 9.3 | 9.4 |
| | | (%) | | | | | | |
| | Loss of chemical | Quantity | 6 | 5.4 | 6.3 | 6.4 | 6.6 | 6.8 |
| | depolymerization | (g) | | | | | | |
| | solution | Ratio | 1.5 | 1.4 | 1.6 | 1.6 | 1.7 | 1.7 |
| | | (%) | | | | | | |

| | | | | | | |
|---|---|---|---|---|---|---|
| Total loss (g) | 175 | 173.4 | 168.3 | 172.4 | 178.6 | 180.8 |
| Total loss rate (%) | 7.8 | 7.7 | 7.5 | 7.7 | 7.9 | 8.0 |

As may be seen from the Table 1 and Table 2, since the extraction solvent and the chemical depolymerization solution of Examples 1 to 6 are all ethylene glycol, compared with Comparative examples 1 to 6, the drying process of removing the extraction solvent before the depolymerization process may be omitted and r-PET having good hue quality and yield may still be obtained. It may be seen that Examples 1 to 6 may simplify the process and be efficient, and may avoid the loss of the extraction solvent during the drying procedure. Therefore, the total loss rate of the extraction solvent and the chemical depolymerization liquid of Examples 1 to 6 is much lower than that of Comparative examples 1 to 6.

Moreover, the extraction solvent and the chemical depolymerization liquid of Examples 1 to 6 may be recycled in a single distillation separation system. Compared with Comparative example 2, which required the use of at least two sets of distillation separation systems to respectively recycle the extraction solvent and the chemical depolymerization liquid, Examples 1 to 6 may reduce equipment costs and operating costs and simplify the recycling procedures.

Based on the above, the method of recycling the polyester fabric of the invention may simplify the process and obtain r-PET having good hue quality and yield by using ethylene glycol as the extraction solvent and the chemical depolymerization solution. Moreover, the method of recycling the polyester fabric of the invention may effectively utilize a single distillation separation system to recycle the extraction solvent and the chemical depolymerization liquid, thus not only reducing the loss of solvent, but also reducing the cost of equipment and operations.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A method of recycling a polyester fabric, comprising:
providing a polyester fabric, wherein the polyester fabric comprises polyethylene terephthalate and dyes;
performing an extraction process to remove the dyes from the polyester fabric, wherein the extraction process comprises using ethylene glycol as an extraction solvent, immersing the polyester fabric in the extraction solvent, and extracting under a temperature of 80° C. to 180° C.;
performing a depolymerization process, wherein the depolymerization process comprises using a chemical depolymerization solution to depolymerize the polyester fabric treated with the extraction process to obtain a product comprising BHET (bis(2-hydroxyethyl terephthalate)), and the chemical depolymerization solution is ethylene glycol;
performing a purification process to remove impurities of the product obtained by the depolymerization process and to obtain a purified BHET; and
performing a solvent recycling process, wherein the solvent recycling process comprises collecting waste solutions of the extraction process and the depolymerization process, and recycling ethylene glycol in the waste solutions by a distillation separating system.

2. The method of recycling the polyester fabric of claim 1, wherein in the extraction process, the polyester fabric is subjected to 1 to 10 extraction steps, and each of the extraction steps is to soak the polyester fabric in a new extraction solvent.

3. The method of recycling the polyester fabric of claim 2, wherein a time of each of the extraction steps is between 20 minutes and 1 hour.

4. The method of recycling the polyester fabric of claim 2, wherein in each of the extraction steps, a weight ratio of the extraction solvent to the polyester fabric is 5 to 10.

5. The method of recycling the polyester fabric of claim 1, wherein the depolymerization process is performed at a temperature of 190° C. to 240° C.

6. The method of recycling the polyester fabric of claim 1, wherein a weight of the chemical depolymerization liquid accounts for 30 wt % to 80 wt % of a total weight of the chemical depolymerization liquid and the polyester fabric treated with the extraction process.

7. The method of recycling the polyester fabric of claim 1, wherein the depolymerization process is performed in an environment containing a catalyst, and the catalyst comprises an organic metal or an ionic liquid.

8. The method of recycling the polyester fabric of claim 7, wherein a weight of the catalyst accounts for 0.3 wt % to 8 wt % of a total weight of the chemical depolymerization liquid and the polyester fabric treated with the extraction process.

9. The method of recycling the polyester fabric of claim 1, wherein a time of the depolymerization process is between 1 hour and 6 hours.

10. The method of recycling the polyester fabric of claim 1, wherein in the solvent recycling process, a recycling rate of the ethylene glycol is greater than 98 wt %.
